SEQUENCE LISTING (9 sequences)

SEQ ID NO: 1 - DNA, 5496 bp, Artificial sequence
  Synthetic
gagctccagcttacacttaattaaacgagacagacaacgcgtacagttcagggcgcgcccccagcacgcagctggtgatctgagaacgggcagactgcctcctcaagtgggtccctgacccctgacccccgagcagcctaactgggaggcaccccccagcaggggcacactgacacctcacacggcagggtattccaacagaccagcagctgagggtcctgtctgttagaaggaaaactaacaaccagaaaggacatctacaccgaaaacccatctgtacatcaccatcctcgaggaccaaaagctagctaaaaccacaaagatggggaaaaaacagaacagaaaaactggaaactctaaaacgcagagcgcctctcctcctccaaaggaacgcagttcctcaccagcaacagaacaaagctggatggagaatgattttgatgagctgagagaagaaggcttcagacgatcaaattactctgagctacgggaggacattcaaaccaaaggcaaagaagttgaaaactttgaaaaaaatttagaagaatgtataactagaataaccaatacagagaagtgcttaaaggagctgatggagctgaaaaccaaggcccgagaactacgtgaagaatgcagaagcctcaggagccgatgcgatcaactggaagaaagggtatcagcaatggaagatgaaatgaatgaaatgaagcgagaagggaagtttagagaaaaaaggatcaaaagaaatgagcaaagcctccaagaaatatgggactatgtgaaaagaccaaatctacgtctgattggtgtacctgaaagtgatgtggagaatggaaccaagttggaaaacactctccaggatattatccaggagaacttccccaatctagcaaggcaggccaacgttcagattcaggaaatacagagaacgccacaaagatactcttcgagaagagcaactccaagacacataattgtcagattcaccaaagttgaaatgaaggaaaaaatgttaagggcagccagagagaaaggtcgggttaccctcaaaggaaagcccatcagactaacagcggatctctcggcagaaaccctacaagccagaagagagtgggggccaatattcaacattcttaaagaaaagaattttcaacccagaatttcatatccagccaaactaagtttcataagtgaaggagagatcaaatactttatagacaagcaaatgttgagagattttgtcaccaccaggcctgccctaaaagagcttctgaaggaagcgctaaacatggaaaggaacaaccggtatcagccgctgcaaaatcatgccaaaatgtaaagaccatcaagactaggaaagatctgcagcaactaatgagcaaaatcaccagctaacatcataatgacaggatcaacttcacacataacaatattaactttaaatataaatggactaaattctgcaatcaagagacacagactggcaagttggataaagagtcaagacccatcagtgtgctgtattcaggaaacccatctcacgtgcagagacacacataggctcaagatcaaaggatggaggaagatttaccaagccaatggaaaacaaaaaaaggcaggggttgcaatcctagtctctgataaaacagactttaaaccaacaaagatcaaaagagacaaagaaggccattacataatggtaaagggatcaattcaacaagaggagctaactatcctaaatatttatgcacccaatacaggagcacccagattcataaagcaagtcctcagtgacctacaaagagacttagactcccacacattaataatgggagactttaacaccccactgtcaacattagacagatcaacgagacagaaagtcaacaaggatacccaggaattgaactcagctctgcaccaagcagacctaatagacatctacagaactctccaccccaaatcaacagaatatacatttttttcagcaccacaccacacctattccaaaattgaccacatagttggaagtaaagctctcctcagcaaatgtaaaagaacagaaattataacaaactatctctcagaccacagtgcaatcaaactagaactcaggattaagaatctcactcaaagccgctcaactacatggaaactgaacaacctgctcctgaatgactactgggtacataacgaaatgaaggcagagatcaagatgttctttgaaaccaacgagaacaaagacaccacataccagaatctctgggacgcattcaaagcagtgtgtagagggaaatttatagcactaaatgcctacaagagaaagcaggaaagatccaaaattgacaccctaacatcacagttgaaagaactagaaaagcaagagcaaacacattcaaaagcaagcagaaggcaagaaataactaaaatcagagcagaactgaaggaaatagagacacaaaaaacccttcaaaaaatcaatgaatccaggagctggttttttgaaaggatcaacaaaattgatagaccgcttgcaagactgatcaagaaaaaaagagagaagaatcaaatagacacgatcaaaaatgataaaggggatatcaccaccgatcccacagaaatacaaactaccatcagagaatactacaaacacctctacgcaaacaagctagaaaatctagaagaaatggatacattcctcgacacatacactctcccaagactaaaccaggaagaagttgaatctctgaatcgaccaataacaggctctgaaattgtggcaataatcaatagtttaccaaccaaaaagagtccaggaccagatggattcacagccgagttctaccagaggtacaaggaggaactggttccattccttctgaaactattccaatcaatagaaaaagagggaatcctccctaactcattttatgaggccagcatcattctgataccaaagccgggcagagacacaaccaaaaaagagaattttagaccaatatccttgatgaacattgatgcaaaaatcctcaacaagatactggcaaaccgaatccagcagcacatcaaaaagctcatccaccatgatcaagtgggcttcatcccggggatgcaaggctggttcaatatacgcaaatcaatcaatgtaatccagcatataaacagagccaaagacaaaaaccacatgattatctcaatagatgcagaaaaagcctttgacaaaattcaacaacccttcatgctaaaaactctcaacaagttaggtattgatgggacgtatttcaaaataataagagctatctatgacaaacccacagccaatatcatactgaatgggcaaaaactggaagcattccctttgaaaaccggcacaagacagggatgccctctctcaccgctcctattcaacatagtgttggaagttctggccagggcaatcaggcaggagaaggagatcaagggtattcaattaggaaaagaggaagtcaaattgtccctgtttgcagacgacatgattgtttatctcgaaaaccccatcgtctcagcccaaaatctcctcaagctgataagcaacttcagcaaagtctcaggatacaaaatcaatgtacaaaaatcacaagcattcttatacaccaacaacagacaaacagagagccaaatcatgggtgaactcccattcacaattgcttcaaagaggatcaaatacctaggaatccaacttacaagggatgtgaaggacctcttcaaggagaactacaaaccactgctcaaggagatcaaagaggagacaaacaaatggaagaacattccatgctcatgggtaggaagaatcaatatcgtgaaaatggccatactgcccaaggtaatttacagattcaatgccatccccatcaagctaccaatgactttcttcacagaattggaaaaaactactttaaagttcatctggaaccaaaaaagggcccgcattgccaagtcaatcctaagccaaaagaacaaagctggaggcatcacactacctgacttcaaactatactacaaggctacagtaaccaaaacagcatggtactggtatcaaaacagagatatagatcaatggaacagaacagagccctcagaaataatgccgcatatctacaactatctgatctttgacaaacctgagaaaaacaagcaatggggaaaggattccctatttaacaagtggtgctgggaaaactggcttgccatttgtagaaagctgaaacttgatcccttccttacaccttatacaaaaatcaattcaagatggatcaaggatttaaacgttaaacctaaaaccataaaaaccctagaagaaaacctaggcattaccattcaggacataggcgtgggcaaggacttcatgtccaaaacaccaaaagccatggcaacaaaagacaaaattgacaaatgggatctaatcaagctaaagagcttctgcacagcaaaagaaactaccatcagagtgaacaggcaacctacaacatgggagaaaatttttgcaacctactcatctgacaaagggctaatatccagaatctacaatgaactcaaacaaatttacaagaaaaaaacaaacaaccccatcaaaaagtgggcgaaggacatgaacagacacttctcaaaagaagacatttatgcagccaaaaaacacatgaagaaatgctcatcatcactggccatcagagaaatgcaaatcaaaaccactatgagatatcatctcacaccagttagaatggcaatcatcaagaagtcaggaaacaacaggtgctggagaggatgcggagaaataggaacacttttacactgttggtgggactgtaaacttgttcaaccattgtggaagtcagtgtggcgattcctcagggatcttgaactagaaataccatttgacccagccatcccattactgggtatatacccaaatgagtataaatcatgctgctataaagacacatgcacacgtatgtttattgcggcactattcacaatagcaaagacttggaaccaacccaaatgtccaacaatgatagactggattaagaaaatgtggcacatatacactatggaatactatgcagccataaaaaatgatgagttcatatcctttgtagggacatggatgaaattggaaaccatcattctcagtaaactatcgcaagaacaaaaaaccaaacaccgcatattctcactcataggtgggaattgaacaatgagatcacatggacacaggaaggggaatatcacactagtgggggactgtggtggggtcgggggaggggggagggatagcattgggagatatacctaatgctagatgacacattagtgggtgcagcgcaccagcatggcacatgaattcatatgtaacttaagctgcacaatgtcgacatgtaccctaaaacttagagtataataaaaaaaaaaaaggatccagcaataagcttataacgcggccgccacaaatccggtacc

SEQ ID NO: 2 - DNA, 5496 bp, Artificial sequence
  Synthetic
gagctccagcttacacttaattaaacgagacagacaacgcgtacagttcagggcgcgcccccagcacgcagctggtgatctgagaacgggcagactgcctcctcaagtgggtccctgacccctgacccccgagcagcctaactgggaggcaccccccagcaggggcacactgacacctcacacggcagggtattccaacagaccagcagctgagggtcctgtctgttagaaggaaaactaacaaccagaaaggacatctacaccgaaaacccatctgtacatcaccatcctcgaggaccaaaagctagctaaaaccgccaccatgggcaagaagcagaaccgcaagaccggcaacagcaagacccagagcgccagccccccccccaaggagcgcagcagcagccccgccaccgagcagagctggatggagaacgacttcgacgagctgcgcgaggagggcttccgccgcagcaactacagcgagctgcgcgaggacatccagaccaagggcaaggaggtggagaacttcgagaagaacctggaggagtgcatcacccgcatcaccaacaccgagaagtgcctgaaggagctgatggagctgaagaccaaggcccgcgagctgcgcgaggagtgccgcagcctgcgcagccgctgcgaccagctggaggagcgcgtgagcgccatggaggacgagatgaacgagatgaagcgcgagggcaagttccgcgagaagcgcatcaagcgcaacgagcagagcctgcaggagatctgggactacgtgaagcgccccaacctgcgcctgatcggcgtgcccgagagcgacgtggagaacggcaccaagctggagaacaccctgcaggacatcatccaggagaacttccccaacctggcccgccaggccaacgtgcagatccaggagatccagcgcaccccccagcgctacagcagccgccgcgccaccccccgccacatcatcgtgcgcttcaccaaggtggagatgaaggagaagatgctgcgcgccgcccgcgagaagggccgcgtgaccctgaagggcaagcccatccgcctgaccgccgacctgagcgccgagaccctgcaggcccgccgcgagtggggccccatcttcaacatcctgaaggagaagaacttccagccccgcatcagctaccccgccaagctgagcttcatcagcgagggcgagatcaagtacttcatcgacaagcagatgctgcgcgacttcgtgaccacccgccccgccctgaaggagctgctgaaggaggccctgaacatggagcgcaacaaccgctaccagcccctgcagaaccacgccaagatgtaaagaccatcaagactaggaaagatctgcagcaactaatgagcaaaatcaccagctaacaccaccatgaccggcagcaacagccacatcaccatcctgaccctgaacatcaacggcctgaacagcgccatcaagcggcaccggctggccagctggatcaagagccaggaccccagcgtgtgctgcatccaggagacccacctgacctgccgggacacccaccggctgaagatcaagggctggcggaagatctaccaggccaacggcaagcagaagaaggccggcgtggccatcctggtgagcgacaagaccgacttcaagcccaccaagatcaagcgggacaaggagggccactacatcatggtgaagggcagcatccagcaggaggagctgaccatcctgaacatctacgcccccaacaccggcgccccccggttcatcaagcaggtgctgagcgacctgcagcgggacctggacagccacaccctgatcatgggcgacttcaacacccccctgagcaccctggaccggagcacccggcagaaggtgaacaaggacacccaggagctgaacagcgccctgcaccaggccgacctgatcgacatctaccggaccctgcaccccaagagcaccgagtacaccttcttcagcgccccccaccacacctacagcaagatcgaccacatcgtgggcagcaaggccctgctgagcaagtgcaagcggaccgagatcatcaccaactacctgagcgaccacagcgccatcaagctggagctgcggatcaagaacctgacccagagccggagcaccacctggaagctgaacaacctgctgctgaacgactactgggtgcacaacgagatgaaggccgagatcaagatgttcttcgagaccaacgagaacaaggacaccacctaccagaacctgtgggacgccttcaaggccgtgtgccggggcaagttcatcgccctgaacgcctacaagcggaagcaggagcggagcaagatcgacaccctgaccagccagctgaaggagctggagaagcaggagcagacccacagcaaggccagccggcggcaggagatcaccaagatccgggccgagctgaaggagatcgagacccagaagaccctgcagaagatcaacgagagccggagctggttcttcgagcggatcaacaagatcgaccggcccctggcccggctgatcaagaagaagcgggagaagaaccagatcgacaccatcaagaacgacaagggcgacatcaccaccgaccccaccgagatccagaccaccatccgggagtactacaagcacctgtacgccaacaagctggagaacctggaggagatggacaccttcctggacacctacaccctgccccggctgaaccaggaggaggtggagagcctgaaccggcccatcaccggcagcgagatcgtggccatcatcaacagcctgcccaccaagaagagccccggccccgacggcttcaccgccgagttctaccagcggtacatggaggagctggtgcccttcctgctgaagctgttccagagcatcgagaaggagggcatcctgcccaacagcttctacgaggccagcatcatcctgatccccaagcccggccgggacaccaccaagaaggagaacttccggcccatcagcctgatgaacatcgacgccaagatcctgaacaagatcctggccaaccggatccagcagcacatcaagaagctgatccaccacgaccaggtgggcttcatccccggcatgcagggctggttcaacatccggaagagcatcaacgtgatccagcacatcaaccgggccaaggacaagaaccacatgatcatcagcatcgacgccgagaaggccttcgacaagatccagcagcccttcatgctgaagaccctgaacaagctgggcatcgacggcacctacttcaagatcatccgggccatctacgacaagcccaccgccaacatcatcctgaacggccagaagctggaggccttccccctgaagaccggcacccggcagggctgccccctgagccccctgctgttcaacatcgtgctggaggtgctggcccgggccatccggcaggagaaggagatcaagggcatccagctgggcaaggaggaggtgaagctgagcctgttcgccgacgacatgatcgtgtacctggagaaccccatcgtgagcgcccagaacctgctgaagctgatcagcaacttcagcaaggtgagcggctacaagatcaacgtgcagaagagccaggccttcctgtacaccaacaaccggcagaccgagagccagatcatgggcgagctgcccttcgtgatcgccagcaagcggatcaagtacctgggcatccagctgacccgggacgtgaaggacctgttcaaggagaactacaagcccctgctgaaggagatcaaggaggacaccaacaagtggaagaacatcccctgcagctgggtgggccggatcaacatcgtgaagatggccatcctgcccaaggtgatctaccggttcaacgccatccccatcaagctgcccatgaccttcttcaccgagctggagaagaccaccctgaagttcatctggaaccagaagcgggcccggatcgccaagagcatcctgagccagaagaacaaggccggcggcatcaccctgcccgacttcaagctgtactacaaggccaccgtgaccaagaccgcctggtactggtaccagaaccgggacatcgaccagtggaaccggaccgagcccagcgagatcatgccccacatctacaactacctgatcttcgacaagcccgagaagaacaagcagtggggcaaggacagcctgttcaacaagtggtgctgggagaactggctggccatctgccggaagctgaagctggaccccttcctgaccccctacaccaagatcaacagccggtggatcaaggacctgaacgtgaagcccaagaccatcaagaccctggaggagaacctgggcatcaccatccaggacatcggcgtgggcaaggacttcatgagcaagacccccaaggccatggccaccaaggacaagatcgacaagtgggacctgatcaagctgaagagcttctgcaccgccaaggagaccaccatccgggtgaaccggcagcccaccacctgggagaagatcttcgccacctacagcagcgacaagggcctgatcagccggatctacaacgagctgaagcagatctacaagaagaagaccaacaaccccatcaagaagtgggccaaggacatgaaccggcacttcagcaaggaggacatctacgccgccaagaagcacatgaagaagtgcagcagcagcctggccatccgggagatgcagatcaagaccaccatgcggtaccacctgacccccgtgcggatggccatcatcaagaagagcggcaacaaccggtgctggcggggctgcggcgagatcggcaccctgctgcactgctggtgggactgcaagctggtgcagcccctgtggaagagcgtgtggcggttcctgcgggacctggagctggagatccccttcgaccccgccatccccctgctgggcatctaccccaacgagtacaagagctgctgctacaaggacacctgcacccggatgttcatcgccgccctgttcaccatcgccaagacctggaaccagcccaagtgccccaccatgatcgactggatcaagaagatgtggcacatctacaccatggagtactacgccgccatcaagaacgacgagttcatcagcttcgtgggcacctggatgaagctggagaccatcatcctgagcaagctgagccaggagcagaagaccaagcaccggatcttcagcctgatcggcggcaactgaacaatgagatcacatggacacaggaaggggaatatcacactagtgggggactgtggtggggtcgggggaggggggagggatagcattgggagatatacctaatgctagatgacacattagtgggtgcagcgcaccagcatggcacatgaattcatatgtaacttaagctgcacaatgtcgacatgtaccctaaaacttagagtataataaaaaaaaaaaaggatccagcaataagcttataacgcggccgccacaaatccggtacc

SEQ ID NO: 3 - DNA, 6059 bp, Homo sapiens
gggggaggagccaagatggccgaataggaacagctccggtctacagctcccagcgtgagcgacgcagaagacggtgatttctgcatttccatctgaggtaccgggttcatctcactagggagtgccagacagtgggcgcaggccagtgtgtgtgcgcaccgtgcgcgagccgaagcagggcgaggcattgcctcacctgggaagcgcaaggggtcagggagttccctttctgagtcaaagaaaggggtgacggtcgcacctggaaaatcgggtcactcccacccgaatattgcgcttttcagaccggcttaagaaacggcgcaccacgagactatatcccacacctggctcggagggtcctacgcccacggaatctcgctgattgctagcacagcagtctgagatcaaactgcaaggcggcaacgaggctgggggaggggcgcccgccattgcccaggcttgcttaggtaaacaaagcagccgggaagctcgaactgggtggagcccaccacagctcaaggaggcctgcctgcctctgtaggctccacctctgggggcagggcacagacaaacaaaaagacagcagtaacctctgcagacttaagtgtccctgtctgacagctttgaagagagcagtggttctcccagcacgcagctggagatctgagaacgggcagacagactgcctcctcaagtgggtccctgactcctgacccccgagcagcctaactgggaggcaccccccagcaggggcacactgacacctcacacggcagggtattccaacagacctgcagctgagggtcctgtctgttagaaggaaaactaacaaccagaaaggacatctacaccgaaaacccatctgtacatcaccatcatcaaagaccaaaagtagataaaaccacaaagatggggaaaaaacagaacagaaaaactggaaactctaaaacgcagagcgcctctcctcctccaaaggaacgcagttcctcaccagcaacggaacaaagctggatggagaatgattttgacgagctgagagaagaaggcttcagacgatcaaattactctgagctacgggaggacattcaaaccaaaggcaaagaagttgaaaactttgaaaaaaatttagaagaatgtataactagaataaccaatacagagaagtgcttaaaggagctgatggagctgaaaaccaaggctcgagaactacgtgaagaatgcagaagcctcaggagccgatgcgatcaactggaagaaagggtatcagcaatggaagatgaaatgaatgaaatgaagcgagaagggaagtttagagaaaaaagaataaaaagaaatgagcaaagcctccaagaaatatgggactatgtgaaaagaccaaatctacgtctgattggtgtacctgaaagtgatgtggagaatggaaccaagttggaaaacactctgcaggatattatccaggagaacttccccaatctagcaaggcaggccaacgttcagattcaggaaatacagagaacgccacaaagatactcctcgagaagagcaactccaagacacataattgtcagattcaccaaagttgaaatgaaggaaaaaatgttaagggcagccagagagaaaggtcgggttaccctcaaaggaaagcccatcagactaacagtggatctctcggcagaaaccctacaagccagaagagagtgggggccaatattcaacattcttaaagaaaagaattttcaacccagaatttcatatccagccaaactaagcttcataagtgaaggagaaataaaatactttatagacaagcaaatgttgagagattttgtcaccaccaggcctgccctaaaagagctcctgaaggaagcgctaaacatggaaaggaacaaccggtaccagccgctgcaaaatcatgccaaaatgtaaagaccatcgagactaggaagaaactgcatcaactaatgagcaaaatcaccagctaacatcataatgacaggatcaaattcacacataacaatattaactttaaatataaatggactaaattctgcaattaaaagacacagactggcaagttggataaagagtcaagacccatcagtgtgctgtattcaggaaacccatctcacgtgcagagacacacataggctcaaaataaaaggatggaggaagatctaccaagccaatggaaaacaaaaaaaggcaggggttgcaatcctagtctctgataaaacagactttaaaccaacaaagatcaaaagagacaaagaaggccattacataatggtaaagggatcaattcaacaagaggagctaactatcctaaatatttatgcacccaatacaggagcacccagattcataaagcaagtcctcagtgacctacaaagagacttagactcccacacattaataatgggagactttaacaccccactgtcaacattagacagatcaacgagacagaaagtcaacaaggatacccaggaattgaactcagctctgcaccaagcagacctaatagacatctacagaactctccaccccaaatcaacagaatatacctttttttcagcaccacaccacacctattccaaaattgaccacatagttggaagtaaagctctcctcagcaaatgtaaaagaacagaaattataacaaactatctctcagaccacagtgcaatcaaactagaactcaggattaagaatctcactcaaagccgctcaactacatggaaactgaacaacctgctcctgaatgactactgggtacataacgaaatgaaggcagaaataaagatgttctttgaaaccaacgagaacaaagacaccacataccagaatctctgggacgcattcaaagcagtgtgtagagggaaatttatagcactaaatgcctacaagagaaagcaggaaagatccaaaattgacaccctaacatcacaattaaaagaactagaaaagcaagagcaaacacattcaaaagctagcagaaggcaagaaataactaaaatcagagcagaactgaaggaaatagagacacaaaaaacccttcaaaaaatcaatgaatccaggagctggttttttgaaaggatcaacaaaattgatagaccgctagcaagactaataaagaaaaaaagagagaagaatcaaatagacacaataaaaaatgataaaggggatatcaccaccgatcccacagaaatacaaactaccatcagagaatactacaaacacctctacgcaaataaactagaaaatctagaagaaatggatacattcctcgacacatacactctcccaagactaaaccaggaagaagttgaatctctgaatagaccaataacaggctctgaaattgtggcaataatcaatagtttaccaaccaaaaagagtccaggaccagatggattcacagccgaattctaccagaggtacatggaggaactggtaccattccttctgaaactattccaatcaatagaaaaagagggaatcctccctaactcattttatgaggccagcatcattctgataccaaagccgggcagagacacaaccaaaaaagagaattttagaccaatatccttgatgaacattgatgcaaaaatcctcaataaaatactggcaaaccgaatccagcagcacatcaaaaagcttatccaccatgatcaagtgggcttcatccctgggatgcaaggctggttcaatatacgcaaatcaataaatgtaatccagcatataaacagagccaaagacaaaaaccacatgattatctcaatagatgcagaaaaagcctttgacaaaattcaacaacccttcatgctaaaaactctcaataaattaggtattgatgggacgtatttcaaaataataagagctatctatgacaaacccacagccaatatcatactgaatgggcaaaaactggaagcattccctttgaaaaccggcacaagacagggatgccctctctcaccgctcctattcaacatagtgttggaagttctggccagggcaatcaggcaggagaaggaaataaagggtattcaattaggaaaagaggaagtcaaattgtccctgtttgcagacgacatgattgtatatctagaaaaccccatcgtctcagcccaaaatctccttaagctgataagcaacttcagcaaagtctcaggatacaaaatcaatgtacaaaaatcacaagcattcttatacaccaacaacagacaaacagagagccaaatcatgggtgaactcccattcgtaattgcttcaaagagaataaaatacctaggaatccaacttacaagggatgtgaaggacctcttcaaggagaactacaaaccactgctcaaggaaataaaagaggacacaaacaaatggaagaacattccatgctcatgggtaggaagaatcaatatcgtgaaaatggccatactgcccaaggtaatttacagattcaatgccatccccatcaagctaccaatgactttcttcacagaattggaaaaaactactttaaagttcatatggaaccaaaaaagagcccgcattgccaagtcaatcctaagccaaaagaacaaagctggaggcatcacactacctgacttcaaactatactacaaggctacagtaaccaaaacagcatggtactggtaccaaaacagagatatagatcaatggaacagaacagagccctcagaaataatgccgcatatctacaactatctgatctttgacaaacctgagaaaaacaagcaatggggaaaggattccctatttaataaatggtgctgggaaaactggctagccatatgtagaaagctgaaactggatcccttccttacaccttatacaaaaatcaattcaagatggattaaagatttaaacgttaaacctaaaaccataaaaaccctagaagaaaacctaggcattaccattcaggacataggcgtgggcaaggacttcatgtccaaaacaccaaaagcaatggcaacaaaagacaaaattgacaaatgggatctaattaaactaaagagcttctgcacagcaaaagaaactaccatcagagtgaacaggcaacctacaacatgggagaaaattttcgcaacctactcatctgacaaagggctaatatccagaatctacaatgaacttaaacaaatttacaagaaaaaaacaaacaaccccatcaaaaagtgggcgaaggacatgaacagacacttctcaaaagaagacatttatgcagccaaaaaacacatgaagaaatgctcatcatcactggccatcagagaaatgcaaatcaaaaccactatgagatatcatctcacaccagttagaatggcaatcattaaaaagtcaggaaacaacaggtgctggagaggatgcggagaaataggaacacttttacactgttggtgggactgtaaactagttcaaccattgtggaagtcagtgtggcgattcctcagggatctagaactagaaataccatttgacccagccatcccattactgggtatatacccaaatgagtataaatcatgctgctataaagacacatgcacacgtatgtttattgcggcactattcacaatagcaaagacttggaaccaacccaaatgtccaacaatgatagactggattaagaaaatgtggcacatatacaccatggaatactatgcagccataaaaaatgatgagttcatatcctttgtagggacatggatgaaattggaaaccatcattctcagtaaactatcgcaagaacaaaaaaccaaacaccgcatattctcactcataggtgggaattgaacaatgagatcacatggacacaggaaggggaatatcacactctggggactgtggtggggtcgggggaggggggagggatagcattgggagatatacctaatgctagatgacacattagtgggtgcagcgcaccagcatggcacatgtatacatatgtaactaacctgcacaatgtgcacatgtaccctaaaacttagagtataataaaaaaaaaaaaaaaaaaaaaaaaaaaaaaaaaaaaa

SEQ ID NO: 4 - DNA, 42 bp, Artificial Sequence
  Synthetic
gcttcataagtgaaggagagatcaagtactttatagacaagc

SEQ ID NO: 5 - DNA, 41 bp, Artificial Sequence
  Synthetic
ggaagtttagagaaaaaaggatcaaaagaaatgagcaaagc

SEQ ID NO: 6 - DNA, 40 bp, Artificial Sequence
  Synthetic
agacacacataggctcaagatcaaaggatggaggaagatc

SEQ ID NO: 7 - DNA, 40 bp, Artificial Sequence
  Synthetic
cagactggcaagttggatcaagagtcaagacccatcagtg

SEQ ID NO: 8 - DNA, 39 bp, Artificial Sequence
  Synthetic
tggactaaattctgcaatcaagagacacagactggcaag

SEQ ID NO: 9 - DNA, 5496 bp, Artificial Sequence
  Synthetic
gagctccagcttacacttaattaaacgagacagacaacgcgtacagttcagggcgcgcccccagcacgcagctggtgatctgagaacgggcagactgcctcctcaagtgggtccctgacccctgacccccgagcagcctaactgggaggcaccccccagcaggggcacactgacacctcacacggcagggtattccaacagaccagcagctgagggtcctgtctgttagaaggaaaactaacaaccagaaaggacatctacaccgaaaacccatctgtacatcaccatcctcgaggaccaaaagctagctaaaaccgccaccatgggcaagaagcagaaccgcaagaccggcaacagcaagacccagagcgccagccccccccccaaggagcgcagcagcagccccgccaccgagcagagctggatggagaacgacttcgacgagctgcgcgaggagggcttccgccgcagcaactacagcgagctgcgcgaggacatccagaccaagggcaaggaggtggagaacttcgagaagaacctggaggagtgcatcacccgcatcaccaacaccgagaagtgcctgaaggagctgatggagctgaagaccaaggcccgcgagctgcgcgaggagtgccgcagcctgcgcagccgctgcgaccagctggaggagcgcgtgagcgccatggaggacgagatgaacgagatgaagcgcgagggcaagttccgcgagaagcgcatcaagcgcaacgagcagagcctgcaggagatctgggactacgtgaagcgccccaacctgcgcctgatcggcgtgcccgagagcgacgtggagaacggcaccaagctggagaacaccctgcaggacatcatccaggagaacttccccaacctggcccgccaggccaacgtgcagatccaggagatccagcgcaccccccagcgctacagcagccgccgcgccaccccccgccacatcatcgtgcgcttcaccaaggtggagatgaaggagaagatgctgcgcgccgcccgcgagaagggccgcgtgaccctgaagggcaagcccatccgcctgaccgccgacctgagcgccgagaccctgcaggcccgccgcgagtggggccccatcttcaacatcctgaaggagaagaacttccagccccgcatcagctaccccgccaagctgagcttcatcagcgagggcgagatcaagtacttcatcgacaagcagatgctgcgcgacttcgtgaccacccgccccgccctgaaggagctgctgaaggaggccctgaacatggagcgcaacaaccgctaccagcccctgcagaaccacgccaagatgtaaagaccatcaagactaggaaagatctgcagcaactaatgagcaaaatcaccagctaacaccaccatgacaggatcaacttcacacataacaatattaactttaaatataaatggcctgaacagcgccatcaagcggcaccggctggccagctggatcaagagccaggaccccagcgtgtgctgcatccaggagacccacctgacctgccgggacacccaccggctgaagatcaagggctggcggaagatctaccaggccaacggcaagcagaagaaggccggcgtggccatcctggtgagcgacaagaccgacttcaagcccaccaagatcaagcgggacaaggagggccactacatcatggtgaagggcagcatccagcaggaggagctgaccatcctgaacatctacgcccccaacaccggcgccccccggttcatcaagcaggtgctgagcgacctgcagcgggacctggacagccacaccctgatcatgggcgacttcaacacccccctgagcaccctggaccggagcacccggcagaaggtgaacaaggacacccaggagctgaacagcgccctgcaccaggccgacctgatcgacatctaccggaccctgcaccccaagagcaccgagtacaccttcttcagcgccccccaccacacctacagcaagatcgaccacatcgtgggcagcaaggccctgctgagcaagtgcaagcggaccgagatcatcaccaactacctgagcgaccacagcgccatcaagctggagctgcggatcaagaacctgacccagagccggagcaccacctggaagctgaacaacctgctgctgaacgactactgggtgcacaacgagatgaaggccgagatcaagatgttcttcgagaccaacgagaacaaggacaccacctaccagaacctgtgggacgccttcaaggccgtgtgccggggcaagttcatcgccctgaacgcctacaagcggaagcaggagcggagcaagatcgacaccctgaccagccagctgaaggagctggagaagcaggagcagacccacagcaaggccagccggcggcaggagatcaccaagatccgggccgagctgaaggagatcgagacccagaagaccctgcagaagatcaacgagagccggagctggttcttcgagcggatcaacaagatcgaccggcccctggcccggctgatcaagaagaagcgggagaagaaccagatcgacaccatcaagaacgacaagggcgacatcaccaccgaccccaccgagatccagaccaccatccgggagtactacaagcacctgtacgccaacaagctggagaacctggaggagatggacaccttcctggacacctacaccctgccccggctgaaccaggaggaggtggagagcctgaaccggcccatcaccggcagcgagatcgtggccatcatcaacagcctgcccaccaagaagagccccggccccgacggcttcaccgccgagttctaccagcggtacatggaggagctggtgcccttcctgctgaagctgttccagagcatcgagaaggagggcatcctgcccaacagcttctacgaggccagcatcatcctgatccccaagcccggccgggacaccaccaagaaggagaacttccggcccatcagcctgatgaacatcgacgccaagatcctgaacaagatcctggccaaccggatccagcagcacatcaagaagctgatccaccacgaccaggtgggcttcatccccggcatgcagggctggttcaacatccggaagagcatcaacgtgatccagcacatcaaccgggccaaggacaagaaccacatgatcatcagcatcgacgccgagaaggccttcgacaagatccagcagcccttcatgctgaagaccctgaacaagctgggcatcgacggcacctacttcaagatcatccgggccatctacgacaagcccaccgccaacatcatcctgaacggccagaagctggaggccttccccctgaagaccggcacccggcagggctgccccctgagccccctgctgttcaacatcgtgctggaggtgctggcccgggccatccggcaggagaaggagatcaagggcatccagctgggcaaggaggaggtgaagctgagcctgttcgccgacgacatgatcgtgtacctggagaaccccatcgtgagcgcccagaacctgctgaagctgatcagcaacttcagcaaggtgagcggctacaagatcaacgtgcagaagagccaggccttcctgtacaccaacaaccggcagaccgagagccagatcatgggcgagctgcccttcgtgatcgccagcaagcggatcaagtacctgggcatccagctgacccgggacgtgaaggacctgttcaaggagaactacaagcccctgctgaaggagatcaaggaggacaccaacaagtggaagaacatcccctgcagctgggtgggccggatcaacatcgtgaagatggccatcctgcccaaggtgatctaccggttcaacgccatccccatcaagctgcccatgaccttcttcaccgagctggagaagaccaccctgaagttcatctggaaccagaagcgggcccggatcgccaagagcatcctgagccagaagaacaaggccggcggcatcaccctgcccgacttcaagctgtactacaaggccaccgtgaccaagaccgcctggtactggtaccagaaccgggacatcgaccagtggaaccggaccgagcccagcgagatcatgccccacatctacaactacctgatcttcgacaagcccgagaagaacaagcagtggggcaaggacagcctgttcaacaagtggtgctgggagaactggctggccatctgccggaagctgaagctggaccccttcctgaccccctacaccaagatcaacagccggtggatcaaggacctgaacgtgaagcccaagaccatcaagaccctggaggagaacctgggcatcaccatccaggacatcggcgtgggcaaggacttcatgagcaagacccccaaggccatggccaccaaggacaagatcgacaagtgggacctgatcaagctgaagagcttctgcaccgccaaggagaccaccatccgggtgaaccggcagcccaccacctgggagaagatcttcgccacctacagcagcgacaagggcctgatcagccggatctacaacgagctgaagcagatctacaagaagaagaccaacaaccccatcaagaagtgggccaaggacatgaaccggcacttcagcaaggaggacatctacgccgccaagaagcacatgaagaagtgcagcagcagcctggccatccgggagatgcagatcaagaccaccatgcggtaccacctgacccccgtgcggatggccatcatcaagaagagcggcaacaaccggtgctggcggggctgcggcgagatcggcaccctgctgcactgctggtgggactgcaagctggtgcagcccctgtggaagagcgtgtggcggttcctgcgggacctggagctggagatccccttcgaccccgccatccccctgctgggcatctaccccaacgagtacaagagctgctgctacaaggacacctgcacccggatgttcatcgccgccctgttcaccatcgccaagacctggaaccagcccaagtgccccaccatgatcgactggatcaagaagatgtggcacatctacaccatggagtactacgccgccatcaagaacgacgagttcatcagcttcgtgggcacctggatgaagctggagaccatcatcctgagcaagctgagccaggagcagaagaccaagcaccggatcttcagcctgatcggcggcaactgaacaatgagatcacatggacacaggaaggggaatatcacactagtgggggactgtggtggggtcgggggaggggggagggatagcattgggagatatacctaatgctagatgacacattagtgggtgcagcgcaccagcatggcacatgaattcatatgtaacttaagctgcacaatgtcgacatgtaccctaaaacttagagtataataaaaaaaaaaaaggatccagcaataagcttataacgcggccgccacaaatccggtacc

(19) United States
(12) Patent Application Publication
(10) Pub. No.: US 2007/0037759 A1
(43) Pub. Date: Feb. 15, 2007

(54) Mammalian retrotransposable elements

(75) Inventors: Prescott Deininger, New Orleans, LA (US); Victoria P. Belancio, New Orleans, LA (US)

Correspondence Address: HOWREY LLP, C/O IP DOCKETING DEPARTMENT, 2941 FAIRVIEW PARK DRIVE, SUITE 200, FALLS CHURCH, VA, 22042-7195, US

(73) Assignee: ADMINISTRATORS OF THE TULANE EDUCATIONAL FUND, THE, New Orleans, LA (US)

(21) Appl. No.: 10/544,458

(22) PCT Filed: Feb. 6, 2004
(86) PCT No.: PCT/US04/03552

§ 371 Date: Jul. 5, 2006

Related U.S. Application Data
(60) Provisional application No. 60/445,945, filed on Feb. 7, 2003.

(51) Int. Cl. A61K 48/00; C12N 15/74

(52) U.S. Cl. 514/44; 435/473

(57) ABSTRACT

The invention relates to a modified retrotransposable element wherein one or more polyadenylation sites have been removed from within the genes of the retrotransposable element, and methods of use thereof. Sequences of optimized retrotransposable element are provided that have polyadenylation sites removed, and may be further modified to optimize codon usage for expression in mammals and to add restriction enzyme sites for ease of use.

### CROSS-REFERENCE TO RELATED APPLICATIONS

This application claims benefit of priority from U.S. Provisional Patent application No. 60/445,945, filed Feb. 7, 2003, the entire contents of which are incorporated by reference herein.

### STATEMENT REGARDING FEDERALLY SPONSORED RESEARCH

This invention was made in part with U.S. Government support under grant number R01 GM45668 awarded by the National Institutes of Health. The U.S. Government has certain rights to this invention.

### FIELD OF THE INVENTION

The present invention relates to the field of molecular biology and methods for altering the genetic material of a cell or organism. More specifically, the invention relates to mammalian retrotransposons.

### BACKGROUND OF THE INVENTION

Various methods have been used to transfer exogenous genetic material into the genomes of cells and organisms including the use of adenovirus, retrovirus and adeno-associated virus vectors. These vectors can be used to produce transgenic animals or for gene therapy applications. However, these vectors do not provide an optimal means of gene transfer due to transient expression and low efficiency of integration.

Naturally occurring mobile genetic elements, known as retrotransposons, are also candidates for gene transfer vehicles. These elements include the non-LTR retrotransposons, among which are found the LINE-like retrotransposons (Long INterspersed Elements) (10). LINE-like elements have been found in animals, insects, plants, protists, and fungi, and include the Drosophila I-element and the maize Cin4 element, among others (31, 32). U.S. Pat. Nos. 5,879,933 and 6,150,160 disclose the use of a particular LINE-like element, the LINE-1 element (L1) retrotransposon for gene delivery. LINE-1 elements are present at about 5×10⁵ copies and represent about 17% of the human genome. Most L1 elements are severely truncated at their 5′ end, and also accumulate other inactivating alterations (11). Thus, there are only about 100 potentially active, full-length L1 copies per human genome (18). There is about one new LINE insertion in every 100 human births, causing about 0.1% of human germ-line disease (9,10).

L1 expression is limited in most differentiated adult cells, but elevated in some cancer cells (1,3,8,13,21), testis and during embryonic development (2). This pattern of expression is explained to some degree by the alterations in the methylation state of the L1 promoter that occur upon malignant transformation (27) and by regulation through the SRY family of transcription factors during embryogenesis (26). However, even when expressed under the very strong CMV promoter in transient assays, very little full-length RNA is produced in cultured cells. Thus, it seems likely that L1 elements have inherent properties limiting their RNA levels, a trait that is likely to have evolved to limit damage to their host genome. The inherent low level of RNA expression from an L1 element and the resulting low efficiency of retrotranspositon make endogenous L1 elements less than optimal for gene insertion and delivery applications. Therefore, there exists a need for a mobile element that demonstrates a high efficiency of retrotransposition.

### SUMMARY OF THE INVENTION

It is therefore an object of the invention to provide a mobile genetic element with a high efficiency of retrotransposition. A principle finding of the present invention is that the elimination of putative polyadenylation (poly(A)) sites in the coding regions of the genes within the retrotransposable element increases the gene expression of the element. The present invention provides a high efficiency retrotransposable element. The retrotransposable element is characterized by a DNA sequence that is optimized for codon usage in mammals and has been engineered to have fewer poly(A) sites within the coding regions of its genes compared to the. endogenous element. Another object of the present invention is a method for increasing the retrotranspostion efficiency of a retrotransposable element by identifying putative poly(A) sites in the coding regions of the element and mutating the putative poly(A) sites to eliminate their functionality while maintaining the original amino acid coding signals.

One aspect of the present invention includes a retrotransposable element, such as a non-LTR retrotransposable element, wherein one or more polyadenylation sites have been removed from within the coding regions of the genes of the retrotransposable element. Another aspect of this invention provides a non-LTR retrotransposable element wherein one or more polyadenylation sites have been removed from within the coding regions of the genes of the retrotransposable element. Another aspect of this invention relates to a retrotransposable element wherein the one or more polyadenylation sites that are removed are selected from the. group consisting of AATAAA, AATACA, AATATA, ACTAAA, AGTAAA, ATTAAA, CATAAA, GATAAA, and TATAAA. Another aspect of this invention relates to a retrotransposable element wherein the predicted polyadenylation site that is removed is the strongest predicted polyadenylation site.

Still another aspect of this invention includes a retrotransposable element derived from a human, a gorilla, or a mouse, including modified L1.3, L1_{RP}, L1spa, or L1Gg-1A elements, wherein one or more polyadenylation sites have been removed from within the coding regions of the genes of the retrotransposable element. A modified retrotransposable element comprising the sequence of SEQ ID NO:1, SEQ ID NO:2, or SEQ ID NO:9 is another aspect of the present invention. A modified active retrotransposable element wherein one or more polyadenylation sites have been removed from within the coding regions of the genes of the retrotransposable element is also an aspect of the present invention.

The present invention also includes a method for increasing the efficiency of retrotransposition comprising identifying one or more putative polyadenylation sites in the coding region of the element and mutating the one or more putative polyadenylation sites to eliminate their functionality while maintaining the original amino acid coding within the genes of said retrotransposable element. Another aspect of the present invention comprises a method for increasing the expression of a gene in a retrotransposable element comprising identifying one or more putative polyadenylation sites in the element and mutagenizing the one or more polyadenylation sites to increase production of mRNA transcripts that comprise sequence located 3′ to the one or more mutagenized polyadenylation sites, while maintaining the original amino acid coding within the genes of the retrotransposable element.

The present invention can be used for insertion of DNA into the genome of a cell or animal. For gene therapy applications, the present invention can be combined with an adenovirus or other viral vector system to achieve high efficiency genomic integration and stable expression of a gene carried by the vector. The present invention can also be used for gene tagging and mutagenesis in mice.

BRIEF DESCRIPTION OF THE SEQUENCES

| | |
|---|---|
| SEQ ID NO: 1 | an optimized retrotransposable element |
| | according to the present invention |
| SEQ ID NO: 2 | an optimized retrotransposable element |
| | according to the present invention |
| SEQ ID NO: 3 | L1.3 GenBank Accession L19088 |
| SEQ ID NO: 4 | mutagenic oligonucleotide Mut 1 |
| SEQ ID NO: 5 | mutagenic oligonucleotide Mut 2 |
| SEQ ID NO: 6 | mutagenic oligonucleotide Mut 3 |
| SEQ ID NO: 7 | mutagenic oligonucleotide Mut 4 |
| SEQ ID NO: 8 | mutagenic oligonucleotide Mut 5 |
| SEQ ID NO: 9 | an optimized retrotransposable element |
| | according to the present invention |

### BRIEF DESCRIPTIONS OF THE DRAWINGS

FIG. 1 shows putative poly(A) sites positioned throughout the coding region of the human, gorilla, and mouse LINE-1 elements. FIG. 1A is a schematic representation of the putative polyadenylation sites identified in the human L1.3, gorilla L1Gg-1A, and mouse L1spa elements. The approximate position of the coding regions of these elements is shown. The genomes were aligned according to the beginning of the open reading frame 1 (ORF1). FIG. 1B is a schematic representation; the positions of the eight single-base variants of the canonical polyadenylation signal AAUAAA in the human L1.3 genome are shown. The strongest predicted polyadenylation site is marked by the asterisk.

FIG. 2 shows utilization of the internal polyA sites upon LINE-1.3 expression in different cell types. FIG. 2A is a schematic representation of the L1.3 expression cassette. The approximate positions of the endogenous L1 promoter, coding region and polyadenylation site (pA), and neomycin resistance gene with an intron (IN) are shown. The SV40 polyadenylation site is located immediately downstream of the L1 cassette. Positions of the putative polyA sites at the top of FIG. 2A are marked by longer (for AAUAAA) or shorter (for AUUAAA) vertical lines. The strongest predicted polyA site is marked by the asterisk. Predicted mRNA species are represented by horizontal lines. Thick black horizontal lines labeled 5′ UTR and neo probe to axon 2 reflect genomic positions of the strand-specific probes used for Northern blot analysis. These strand-specific probes were generated by Maxiscript in vitro transcription kit (Ambion) from T7 promoter included in the primer that was used to produce the template DNA. FIG. 2B represents a northern blot detection of L1.3 RNA species produced by wild type (wt) element and one with the mutation in the strongest predicted polyA site (Mut) in NIH 3T3, Ntera2, HeLa, and chicken cells with strand specific 5′ UTR probe (four T75 flasks with 3 to 4.5×10⁶ cells were transfected with the L1.3 expression cassette by lipofectamine (Invitrogen). 24 hours post transfections total mRNA was harvested, polyA selected and precipitated (PolyATract mRNA isolation system, Promega), and run on an agarose-formaldehyde gel). FL1.3 is full-length L1.3 mRNA, enFL1.3 is endogenous L1.3 full-length mRNA. Long horizontal arrows correspond to the positions of the molecular weight RNA marker (Invitrogen). RNA bands that are described specifically herein are marked.

FIG. 3 shows sequence analysis of the 3′ end of the L1.3 RNA species identifying that both canonical and noncanonical internal polyanylation sites are functional. 3′ RACE (Clontech) of the polyA selected total mRNA from chicken fibroblast transiently transfected with the wildtype or mut L1.3 expression vector described in FIG. 2. The upstream primer used in the PCR step corresponds to the position 1342-1359 of the L1.3 sense strand. 1 kb DNA ladder (NEBiolabs, lane 1), 3′ RACE reaction of the wt (lane 2) and mut (lane 3) L1.3 without reverse transcriptase (RT), 3′ RACE reaction of the wt (lane 4) and mut (lane 5) L1.3 with the L1.3 specific primer. The band corresponding to the strongest predicted polyA site identified by the Northern blot is labeled 3. When compared to lane 4, lane 5 demonstrates increased levels of longer transcripts with the removal of the strongest polyadenylation site. Genomic location and the hexonucleotide sequence of the identified functional polyA sites are shown next to their positions on the gel.

FIG. 4 shows the DNA. sequence (SEQ ID NO:1) following optimization of the L1 element to remove the polyadenylation signals within the coding regions.

FIG. 5 shows the DNA sequence (SEQ ID NO:2) of the retrotransposable element of the present invention following codon optimization of the DNA sequence shown in FIG. 4.

### DETAILED DESCRIPTION OF THE INVENTION

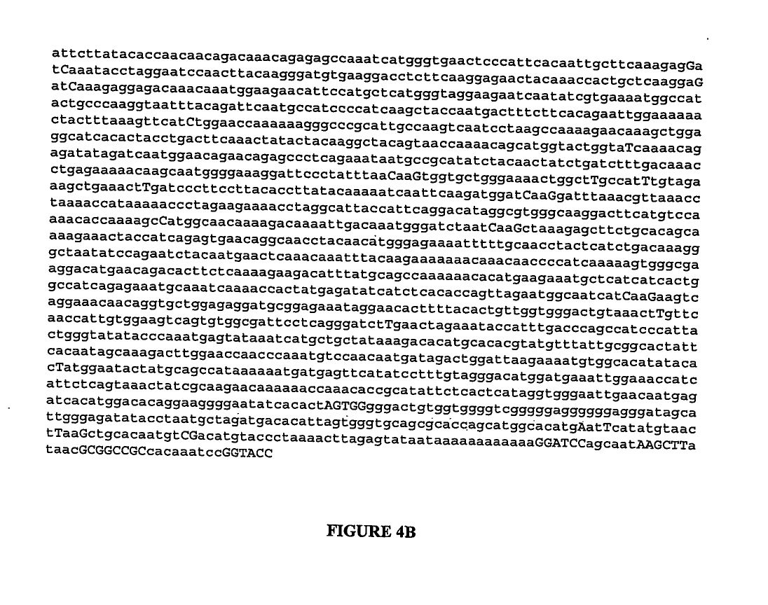

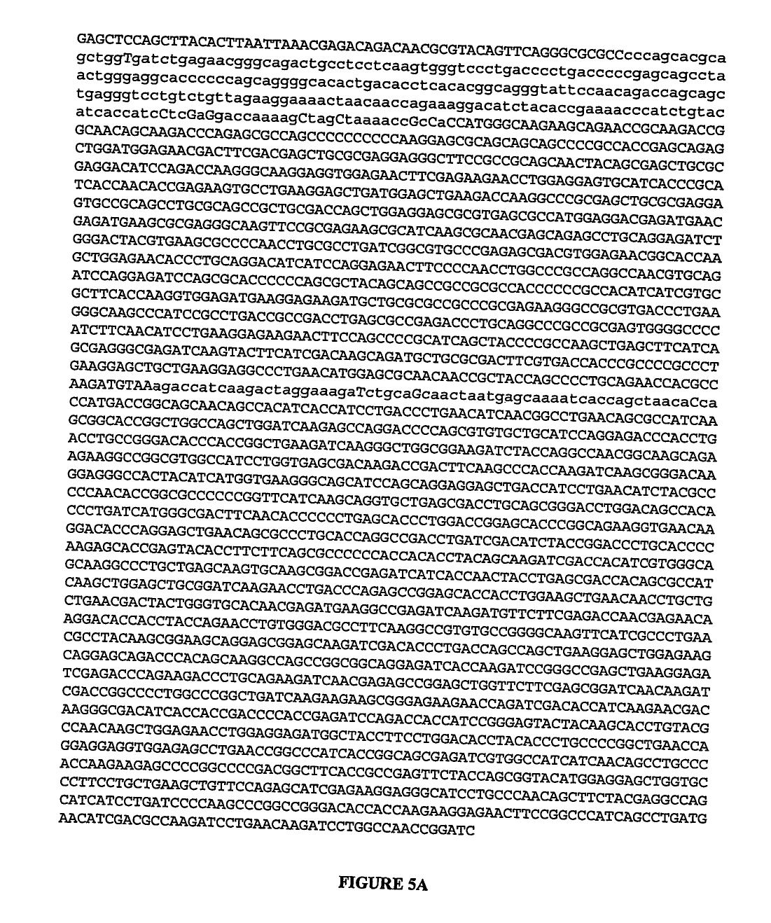

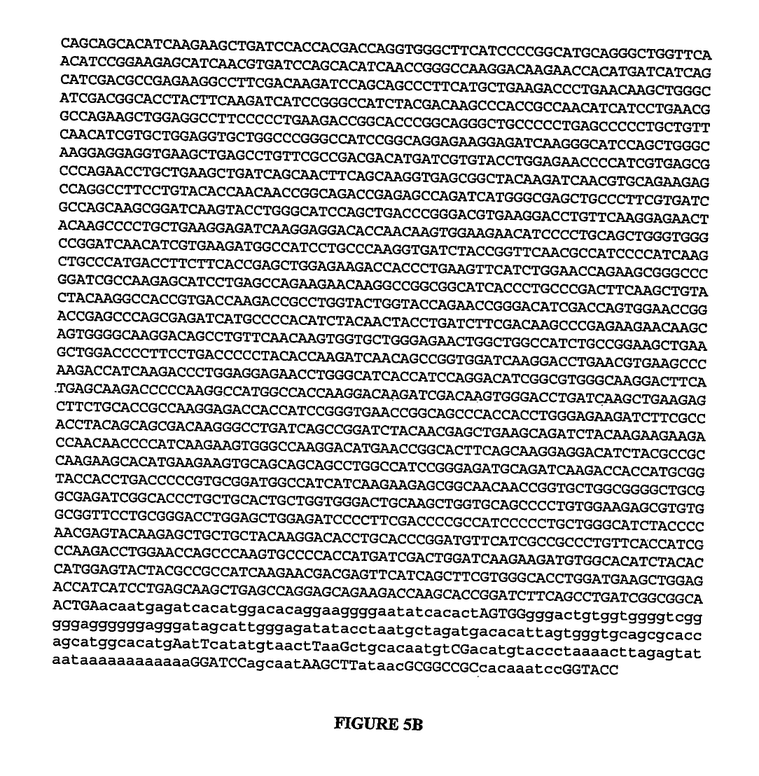

The present invention is an endogenous retrotransposable element optimized to contain mutations that have eliminated polyadenylation sites within the coding regions of the genes within the element and have optimized the codon usage for protein translation in mammals. The retrotransposable element that is being optimized may be a non-LTR element, such as a LINE-like element. Examples of such elements include, but are not limited to, L1.3 (30; SEQ ID NO:3; GenBank Accession L19088), L1_{RP} (33; GenBank Accession AF148856), L1Gg-1a (35; GenBank Accession AF036235), and L1spa (34; GenBank Accession AF016099). These mutations would therefore increase the level of production of the retrotransposable element from the DNA sequence, and result in an increased efficiency of retrotransposition compared to the non-mutated element. As a preferred embodiment of the invention, the retrotransposable element is an optimized mammalian L1 element, such as an optimized L1.3 or an optimized L1_{RP}. As a further preferred embodiment, the retrotransposable element is encoded by the DNA sequence shown in FIG. 5A and FIG. 5B [SEQ ID NO:2]. An alternative embodiment of the present invention is the DNA sequence shown in FIG. 4A and FIG. 4B, [SEQ ID NO:1], where the polyadenylation sites have been removed from the coding regions of the L1 element's genes, but the amino acid coding sequences have not been optimized for codon usage in mammals. The DNA sequence of SEQ ID NO:9 is another preferred embodiment of the invention.

### EXAMPLES

These examples are presented for purposes of illustration only and are not intended to limit the scope of the invention in any way. Techniques common to the field of molecular biology were used.

Identification of PolyA Sites

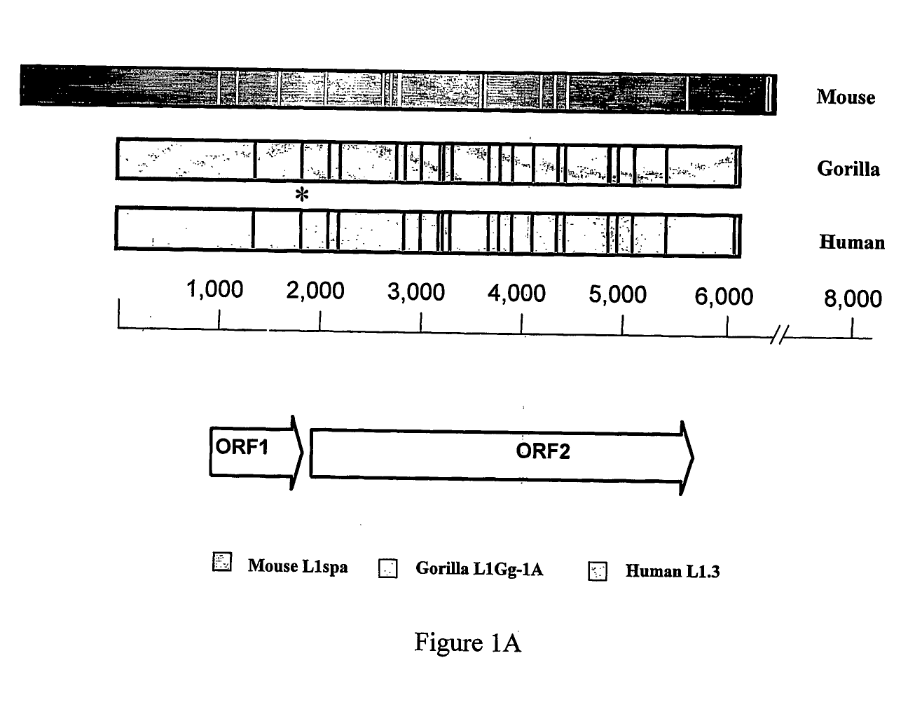

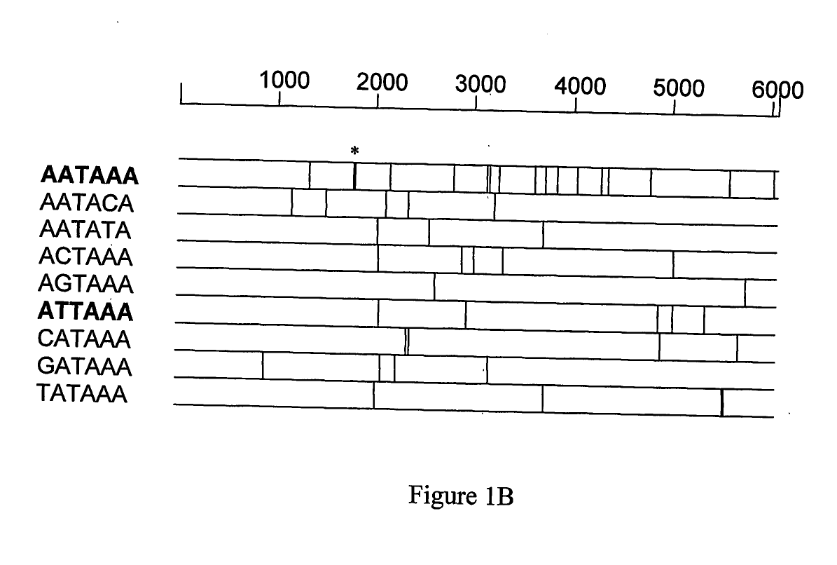

The L1 genome has a large number of potential hexanucleotide, poly(A) signals (FIG. 1A) in its sense strand, yet very few in the antisense direction. This is the opposite pattern seen in most cellular genes, as selection apparently limits the number of potential poly(A)sites that could cause premature termination of transcription. A functional polyadenylation signal required for transcriptional termination by polymerase II (PolII) consists of three sequence elements that determine the exact site of the cleavage and polyadenylation. The most conserved of the three is the AATAAA hexanucleotide. Examples of other hexanucleotide polyadenylation signals include AATACA, AATATA, ACTAAA, AGTAAA, ATTAAA, CATAAA, GATAAA, TATAAA. The actual cleavage/polyadenylation site is located 11-23 nt 3′ of the hexamer and 10-30 nt upstream of a GU- or U-rich region, the third element of the polyadenylation signal (17). A program that predicts the relative strength of poly(A) sites based on their sequence context (polyadq, (23)), predicted that several of these sites were potentially fairly strong. In fact, they were predicted to be stronger than the relatively weak poly(A) site normally found at the 3′ end of the L1 element and one is predicted to be stronger than the SV40 poly(A) site used in the vector. Furthermore, the positions of these polyadenylation sites were strikingly conserved between active human and full-length gorilla elements, and show similar numbers and approximate locations in active mouse elements (FIG. 1A). Thus, the abundance, predicted strength, and conservation, suggest that these sites may limit the amount of full-length L1 RNA produced.

There are a number of examples where alternative polyadenylation has been involved in producing variant mRNAs in a tissue- or development-specific manner (14,19,24,25,28,29), with testis being a common source for alternative polyadenylation (6). Such regulation usually involves multiple weak poly(A)sites, with the strongest polyadenylation site being located at the most 3′ end. However, polyadenylation sites utilized in this way are almost always positioned in the introns or 3′ UTRs of the regulated genes (6), rather than in their coding regions as seen for L1 (FIG. 1a).

Expression of L1 mRNA

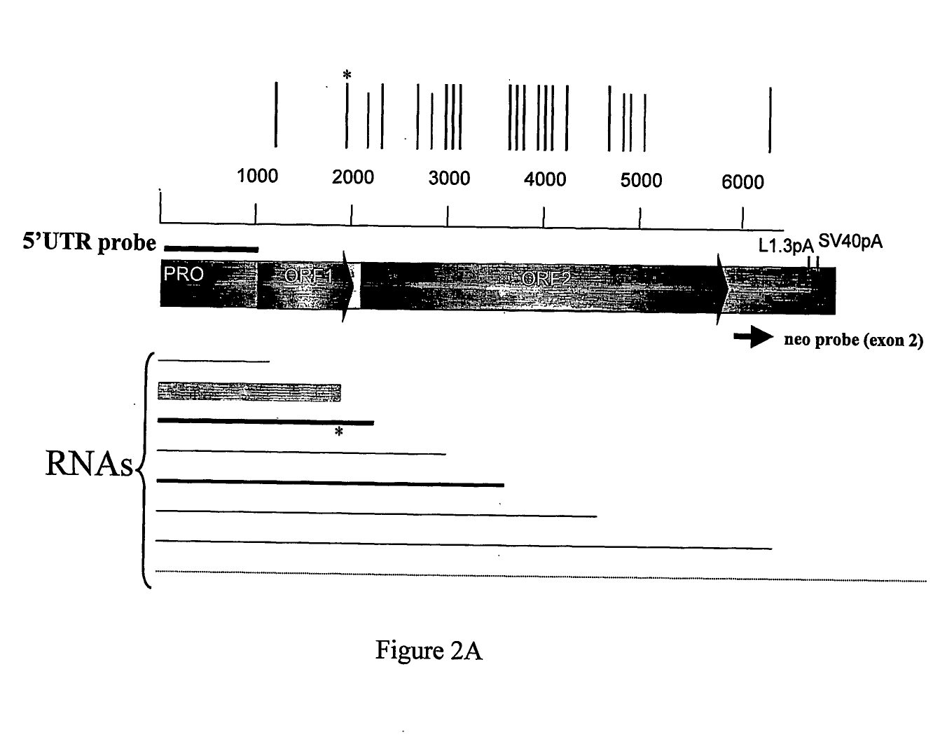

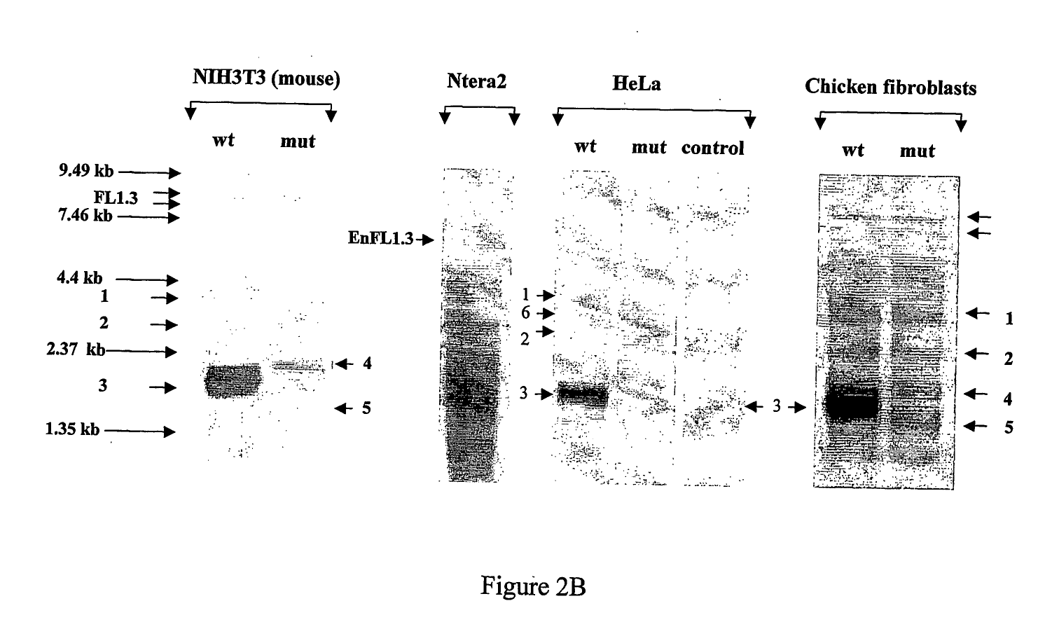

The active L1 element, L1.3 (30) [SEQ ID NO:3] was transiently expressed in mouse NIH 3T3 cells using the CMV promoter to improve expression from a selectable L1 vector-that has been shown to undergo efficient retrotransposition-(5,16). This vector includes an intron and a selectable marker in the 3′ non-coding region. Expression in mouse cells allowed the use of probes from the 5′ end of the human L1 element to detect RNAs produced from this transfected L1 element without background from endogenous L1 RNAs (12,22). The 5′-UTR strand-specific probe was used (FIG. 2A), because it would permit identification of any RNAs truncated by polyadenylation at the internal poly(A)sites (FIG. 2B). This assay detected two high molecular weight polyadenylated L1.3-specific RNAs enriched in the polyA-selected fraction that migrated between 7.4 and 9.4 kb (FL1.3, FIG. 2B). A doublet with the identical molecular weights was detected in the polyA-selected fraction with a strand-specific RNA probe to the neomycin selectable marker located at the 3′ end of the L1.3 construct (FIG. 2A, and data not shown). The presence of the doublet would be consistent with full-length transcripts with either inefficient splicing of the intron located in the neomycin gene (resulting in the 7.7 kb spliced and 8.6 kb unspliced mRNAs) or the alternative usage of the L1.3 3′ UTR polyadenylation site (15) and the SV40 poly(A) site located immediately downstream.

In addition to these relatively weak bands for apparently full-length L1 RNAs, multiple faster-migrating L1.3-specific polyadenylated RNAs were detected that were much more abundant. The sizes of these RNAs roughly corresponded to the positions of many of the putative polyadenylation sites identified in the L1.3 genome (bands #1 through #3, FIG. 2B). The bands were absent from the polyA fraction, and they were not detected by the neomycin strand-specific probe which should be located downstream of their termination sites (data not shown). The strongest band, # 3 (FIG. 2B), corresponds in size to an RNA that would be predicted to terminate in the vicinity of the strongest predicted polyadenylation site.

The strongest predicted polyadenylation signal was mutated to demonstrate the use of this site in formation of the major RNA species. Two point mutations were introduced in the most conserved AATAAA element of the polyadenylation site in order to avoid any residual activity (20). As expected, wild-type band #3 completely disappeared upon mutation (FIG. 2B), indicating that the destroyed polyadenylation site was solely responsible for the production of this band. This confirmed that at least one of the internal polyadenylation sites is functional, and contributes to limiting the full-length L1.3 expression. Removal of the strongest polyadenylation site resulted in the more efficient utilization of the polyadenylation sites located 5′ and 3′ relative to its position (bands #4 and 5, FIG. 2B). In this analysis, the amount of the full-length L1.3 mRNA (FL1.3 combined, FIG. 2B) remained similar in the wt and mutant at an average of 2% of the total RNA. It has been previously observed that the relative activity of any given polyadenylation site varies depending on the presence of other poly(A) sites in its vicinity. Thus, each internal polyadenylation site present in the L1.3 genome alone may have a minor effect on limiting the L1.3 expression, but the sum of them may result in a 50-fold decrease in the amount of the full-length mRNA that is produced in the NIH 3T3 cells.

In order to compare the behavior of the internal polyadenylation sites of the human L1.3 RNA expressed in the mouse cells to the endogenous L1 expression, total polyadenylated RNAs was isolated from human Ntera2 cells and HeLa cells. Ntera2 cells have been demonstrated to express relatively high levels of full-length L1 RNA, while HeLa cells produce very little (FIG. 2B). The full-length RNA is of a different length because the endogenous L1 does not include the extra selectable marker region in our transfected element. However, there is a similarity in the endogenous lower molecular weight bands present in Ntera2 cells to those from expression of the L1.3 vector in NIH 3T3 cells. The differences are likely to represent variation in the polyadenylation sites present within the full-length L1 elements being expressed from various endogenous loci. Comparing the band distribution in transiently transfected and non-transfected HeLa cells to those in the mouse cells (FIG. 2B), a similar pattern was observed in both cell lines with the exception of band #6 that was detected in HeLa but not in NIH 3T3 cells, suggesting possible species or cell type-specific differences in pA-site utilization.

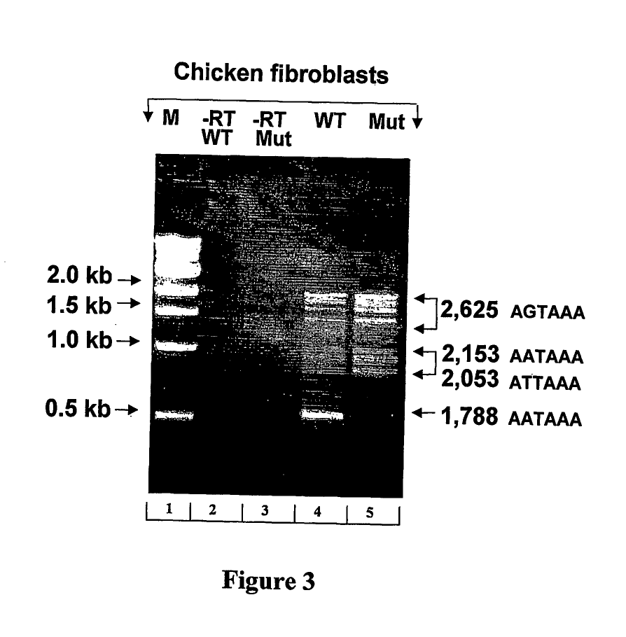

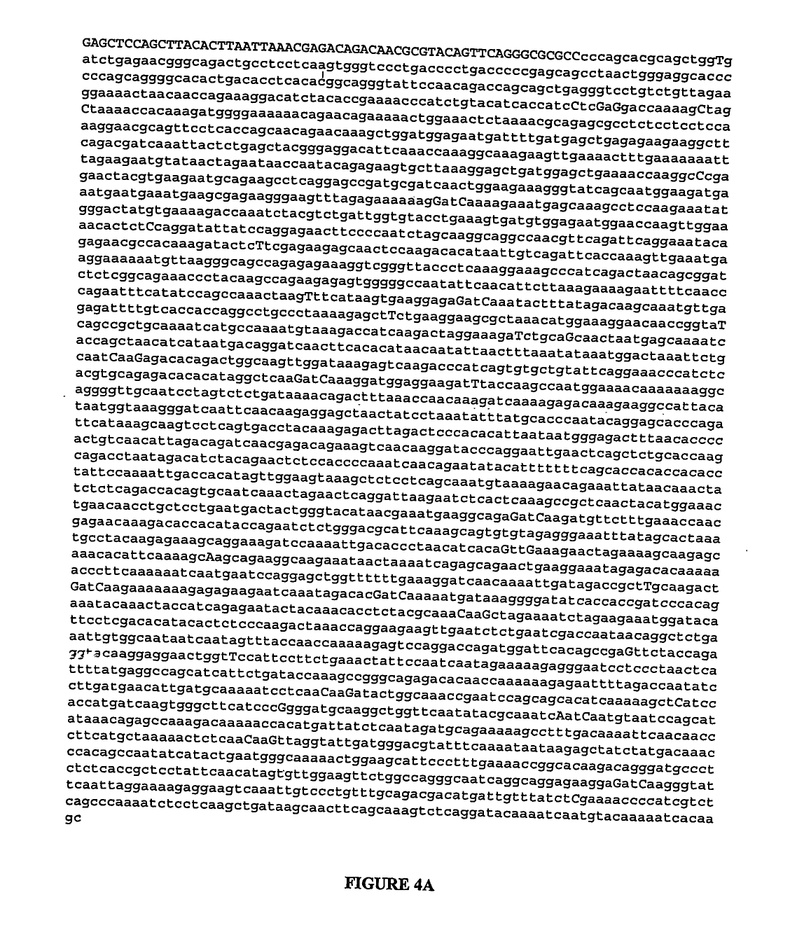

To further confirm the use of specific internal L1 polyadenylation sites, 3′RACE was performed on the poly A-selected mRNAs from the chicken fibroblasts. A chicken cell line was used for this assay to prevent interference from any endogenous, mammalian, L1 RNAs. Northern blot analysis demonstrated that expression of the human L1 vector produced similar RNA species in chicken cells (FIG. 2B). The banding pattern is identical, with only some quantitative differences. This suggests that, like with the mouse cells, there may be species or tissue-specific differences in the utilization of the polyadenylation sites. Employing the 3′ RACE to the region expected to produce the major truncated L1 RNA products in the northern blots showed a pattern consistent with the northern blot results (FIG. 3). The band corresponding to the strongest polyadenylation site in the wildtype, at position 1788, disappeared upon mutagenesis. Sequence analysis of several of the bands revealed the usage of the canonical AATAAA, the next most commonly utilized variant ATTAAA, as well as other non-canonical internal poly(A) sites with one base variation from the AATAAA (FIG. 3).

Generation of High Efficiency L1 elements

Elimination of one or more polyadenylation sites within the coding regions of the L1 element would yield a retrotransposable element that would demonstrate an increased efficiency of retrotransposition, given the expected increase in the number of full-length transcripts. The coding sequence of the L1 DNA was altered with base changes that eliminated the primary polyadenylation sites, but still retained the original amino acid coding. The DNA sequence of the L1 element optimized to remove polyadenylation signals within the coding regions is shown in FIGS. 4A and 4B. In addition, the L1 element optimized to reduce internal polyadenylation was also optimized for codon usage in mammals. Codon bias analysis and optimization was conducted using the software program Primo Optimum 3.4 (ChangBioscience). The DNA sequence of the resultant high efficiency retrotransposable element is shown in FIGS. 5A and 5B. An alternative DNA sequence (SEQ ID NO:9) represents another optimized L1 element design that allows for engineering of convenient restriction enzyme sites and retains sequences at the start of ORF2 that are thought to be important for translation of the gene. For the elements shown in FIG. 4, FIG. 5, and SEQ ID NO:9, a promoter must be inserted 5′ to the DNA sequences in order to drive expression of the element.

Assay of Retrotransposition Potential

Mutants were compared to wild-type using the standard retrotransposition experiment cassette in HeLa cells. 5×10⁵ HeLa cells per 75 cm² cell culture flask (Corning Inc.) were transfected with 0.3 or 0.4 μg of L1.3 wildtype, and an expression cassette consisting of either 1 Mut (with the strongest polyadenylation site removed) or 5 Mut (polyadenylation sites removed by use of mutagenic oligonucleotides of SEQ ID NO:4 through SEQ ID NO:8) by lipofectamine (3 or 4 ml of Plus reagent, 1.5 or 1 μl of lipofectamine, Invitrogen) 16 to 18 hours after plating. At three hours post transfection, the transfection cocktail in serum free DMEM was replaced with MEM (GIBCO), 10% fetal bovine serum (FBS, GIBCO). 24 hours later media was replaced with media containing G418 sulphate (Mediatech, Inc. 440 μg/ml) or Hygromycin B (Invitrogen, 250 mg/ml) selection. The cells were fed every 3-4 days. After 14 days of growing under selection the cells were fixed and stained (0.2% crystal violet, 2.5% isopropanol, 5% acetic acid).

The colonies from the assays were counted with an automated colony counter and are presented in the first two columns of table 1 and table 2. Independent experiments were performed on separate days with differing transfection conditions that resulted in high levels of variation between experiments. In order to compare experiments more effectively, we normalized each experiment to the average number of colonies produced from the wild-type construct for that experiment (presented in the normalized columns (N)). The average and standard deviation for each normalized column is presented. The data were analyzed by ANOVA either as each column being a single experiment, or allowing each experiment to be considered independently. All experiments support a modest but statistically significant stimulation of retrotransposition by the polyadenylation mutations. Each experiment included parallel controls for transfection by using selection for the constitutive hygromycin cassette on the backbone of the L1 retrotransposition plasmid.

TABLE 1

| | WT | 1 Mut | WT (N) | 1 Mut (N) |
|---|---|---|---|---|
| Expt. 1 | 34 | 63 | 121 | 225 |
| | 22 | 70 | 79 | 250 |
| | 28 | 124 | 100 | 443 |
| Expt. 2 | 15 | 20 | 196 | 261 |
| | 3 | 16 | 39 | 209 |
| | 5 | 27 | 65 | 352 |
| Expt. 3 | 15 | 21 | 107 | 150 |
| | 17 | 8 | 121 | 57 |
| | 10 | 0 | 71 | 0 |
| Expt. 4 | 69 | 156 | 83 | 187 |
| | 91 | 138 | 109 | 166 |
| | 90 | 39 | 108 | 47 |
| Expt. 5 | 64 | 95 | 78 | 116 |
| | 82 | 108 | 100 | 132 |
| | 100 | 102 | 122 | 124 |
| Expt. 6 | 37 | 58 | 107 | 167 |
| | 30 | 47 | 87 | 136 |
| | 37 | 62 | 107 | 179 |
| Average | 42 | 64 | 100 | 175 |
| Std. Dev. | | | 33 | 106 |

| | | |
|---|---|---|
| P-Value | ANOVA w/ replicates | 0.0005 |
| P-value | ANOVA as single experiment. | 0.0053 |

TABLE 2

| | Wt | 5 Mut | WT (N) | 5 Mut (N) |
|---|---|---|---|---|
| Expt. 1 | 64 | 266 | 78 | 324 |
| | 82 | 107 | 100 | 130 |
| | 100 | 146 | 122 | 178 |
| Expt. 2 | 37 | 43 | 107 | 123 |
| | 30 | 60 | 87 | 173 |
| | 37 | 62 | 107 | 177 |
| Expt. 3 | 5 | 14 | 50 | 140 |
| | 14 | 11 | 140 | 110 |
| | 11 | 18 | 110 | 180 |
| Expt. 4 | 5 | 12 | 125 | 300 |
| | 3 | 6 | 75 | 150 |
| | 4 | 6 | 100 | 150 |
| Average | 32.7 | 62.5 | 100.0 | 177.9 |
| Std. Dev. | | | | 67.0 |

| | | |
|---|---|---|
| P-Value | ANOVA w/replicates | 0.003 |
| P-Value | ANOVA as single experiment. | 0.001 |

### LIST OF REFERENCES

Citation or identification of any reference in any section of this application shall not be construed as an admission that such reference is available as prior art to the present invention.
1. Asch, H. L., E. Eliacin, T. G. Fanning, J. L. Connolly, G. Bratthauer, and B. B. Asch. 1996. Comparative expression of the LINE-1 p40 protein in human breast carcinomas and normal breast tissues. Oncol. Res. 8:239-247.
2. Branciforte, D. and S. L. Martin. 1994. Developmental and cell type specificity of LINE-1 expression in mouse testis: implications for transposition. Mol. Cell Biol. 14:2584-2592.
3. Bratthauer, G. L., R. D. Cardiff, and T. G. Fanning. 1994. Expression of LINE-1 retrotransposons in human breast cancer. Cancer 73:2333-2336.
4. Deininger, P. L. and M. A. Batzer. 1999. Alu repeats and human disease. Mol. Genet. Metab 67:183-193.
5. Dombroski, B. A., S. L. Mathias, E. Nanthakumar, A. F. Scott, and H. H. Kazazian, Jr. 1991. Isolation of an active human transposable element. Science 254:1805-1808.
6. Edwalds-Gilbert, G., K. L. Veraldi, and C. Milcarek. 1997. Alternative poly(A) site selection in complex transcription units: means to an end? Nucleic Acids Res. 25:2547-2561.
7. Esnault, C., J. Maestre, and T. Heidmann. 2000. Human LINE retrotransposons generate processed pseudogenes. Nat. Genet. 24:363-367.
8. Florl, A. R., R. Lower, B. J. Schmitz-Drager, and W. A. Schulz. 1999. DNA methylation and expression of LINE-1 and HERV-K provirus sequences in urothelial and renal cell carcinomas. Br. J. Cancer 80:1312-1321.
9. Kazazian, H. H., Jr. 1998. Mobile elements and disease. Curr. Opin. Genet. Dev. 8:343-350.
10. Kazazian, H. H., Jr. and J. V. Moran. 1998. The impact of L1 retrotransposons on the human genome. Nat. Genet 19:19-24
11. Lander, E. S., L. M. Linton, B. Birren, C. Nusbaum, M. C. Zody, J. Baldwin, K. Devon, K. Dewar, M. Doyle, W. FitzHugh, R. Funke, D. Gage, K. Harris, A. Heaford, J. Howland, L. Kann, J. Lehoczky, R. LeVine, P. McEwan, K. McKernan, J. Meldrim, J. P. Mesirov, C. Miranda, W. Morris, J. Naylor, C. Raymond, M. Rosetti, R. Santos, A. Sheridan, C. Sougnez, N. Stange-Thomann, N. Stojanovic, A. Subramanian, D. Wyman, J. Rogers, J. Sulston, R. Ainscough, S. Beck, D. Bentley, J. Burton, C. Clee, N. Carter, A. Coulson, R. Deadman, P. Deloukas, A. Dunham, I. Dunham, R. Durbin, L. French, D. Grafham, S. Gregory, T. Hubbard, S. Humphray, A. Hunt, M. Jones, C. Lloyd, A. McMurray, L. Matthews, S. Mercer, S. Milne, J. C. Mullikin, A. Mungall, R. Plumb, M. Ross, R. Shownkeen, S. Sims, R. H. Waterston, R. K. Wilson, L. W. Hillier, J. D. McPherson, M. A. Marra, E. R. Mardis, L. A. Fulton, A. T. Chinwalla, K. H. Pepin, W. R. Gish, S. L. Chissoe, M. C. Wendl, K. D. Delehaunty, T. L. Miner, A. Delehaunty, J. B. Kramer, L. L. Cook, R. S. Fulton, D. L. Johnson, P. J. Minx, S. W. Clifton, T. Hawkins, E. Branscomb, P. Predki, P. Richardson, S. Wenning, T. Slezak, N. Doggett, J. F. Cheng, A. Olsen, S. Lucas, C. Elkin, E. Uberbacher, M. Frazier, R. A. Gibbs, D. M. Muzny, S. E. Scherer, J. B. Bouck, E. J. Sodergren, K. C. Worley, C. M. Rives, J. H. Gorrell, M. L. Metzker, S. L. Naylor, R. S. Kucherlapati, D. L. Nelson, G. M. Weinstock, Y. Sakaki, A. Fujiyama, M. Hattori, T. Yada, A. Toyoda, T. Itoh, C. Kawagoe, H. Watanabe, Y. Totoki, T. Taylor, J. Weissenbach, R. Heilig, W. Saurin, F. Artiguenave, P. Brottier, T. Bruls, E. Pelletier, C. Robert, P. Wincker, A. Rosenthal, M. Platzer, G. Nyakatura, S. Taudien, A. Rump, H. M. Yang, J. Yu, J. Wang, G. Y. Huang, J. Gu, L. Hood, L. Rowen, A. Madan, S. Z. Qin, R. W. Davis, N. A. Federspiel, A. P. Abola, M. J. Proctor, R. M. Myers, J. Schmutz, M. Dickson, J. Grimwood, D. R. Cox, M. V. Olson, R. Kaul, C. Raymond, N. Shimizu, K. Kawasaki, S. Minoshima, G. A. Evans, M. Athanasiou, R. Schultz, B. A. Roe, F. Chen, H. Q. Pan, J. Ramser, H. Lehrach, R. Reinhardt, W. R. McCombie, M. de la Bastide, N. Dedhia, H. Blocker, K. Hornischer, G. Nordsiek, R. Agarwala, L. Aravind, J. A. Bailey, A. Bateman, S. Batzoglou, E. Birney, P. Bork, D. G. Brown, C. B. Burge, L. Cerutti, H. C. Chen, D. Church, M. Clamp, R. R. Copley, T. Doerks, S. R. Eddy, E. E. Eichler, T. S. Furey, J. Galagan, J. G. R. Gilbert, C. Harmon, Y. Hayashizaki, D. Haussler, H. Hermjakob, K. Hokamp, W. H. Jang, L. S. Johnson, T. A. Jones, S. Kasif, A. Kaspryzk, S. Kennedy, W. J. Kent, P. Kitts, E. V. Koonin, I. Korf, D. Kulp, D. Lancet, T. M. Lowe, A. McLysaght, T. Mikkelsen, J. V. Moran, N. Mulder, V. J. Pollara, C. P. Ponting, G. Schuler, J. R. Schultz, G. Slater, A. F. A. Smit, E. Stupka, J. Szustakowki, D. Thierry-Mieg, J. Thierry-Mieg, L. Wagner, J. Wallis, R. Wheeler, A. Williams, Y. I. Wolf, K. H. Wolfe, S. P. Yang, R. F. Yeh, F. Collins, M. S. Guyer, J. Peterson, A. Felsenfeld, K. A. Wetterstrand, A. Patrinos, and M. J. Morgan. 2001. Initial sequencing and analysis of the human genome. Nature 409:860-921.
12. Loeb, D. D., R. W. Padgett, S. C. Hardies, W. R. Shehee, M. B. Comer, M. H. Edgell, and C. A. Hutchison, III. 1986. The sequence of a large L1Md element reveals a tandemly repeated 5′ end and several features found in retrotransposons. Mol. Cell Biol. 6:168-182.
13. Martin, S. L. 1991. Ribonucleoprotein particles with LINE-1 RNA in mouse embryonal carcinoma cells. Mol. Cell Biol. 11:4804-4807.
14. Martincic, K., R. Campbell, G. Edwalds-Gilbert, L. Souan, M. T. Lotze, and C. Milcarek. 1998. Increase in the 64-kDa subunit of the polyadenylation/cleavage stimulatory factor during the G0 to S phase transition. Proc. Natl. Acad. Sci. U.S.A 95:11095-11100.
15. Moran, J. V., R. J. DeBerardinis, and H. H. Kazazian, Jr. 1999. Exon shuffling by L1 retrotransposition. Science 283:1530-1534.
16. Moran, J. V., S. E. Holmes, T. P. Naas, R. J. DeBerardinis, J. D. Boeke, and H. H. Kazazian, Jr. 1996. High frequency retrotransposition in cultured mammalian cells. Cell 87:917-927.
17. Proudfoot, N. 1991. Poly(A) signals. Cell 64:671-674.
18. Sassaman, D. M., B. A. Dombroski, J. V. Moran, M. L. Kimberland, T. P. Naas, R. J. DeBerardinis, A. Gabriel, G. D. Swergold, and H. H. Kazazian, Jr. 1997. Many human L1 elements are capable of retrotransposition [see comments]. Nat. Genet. 16:37-43.
19. Setzer, D. R., M. Mcgrogan, J. H. Nunberg, and R. T. Schimke. Size heterogeneity in the 3′ eng of dihydrofolate reductase messenger RNA in mouse cells. Cell 22, 361-370. 1980.
20. Sheets, M. D., S. C. Ogg, and M. P. Wickens. 1990. Point mutations in AAUAAA and the poly (A) addition site: effects on the accuracy and efficiency of cleavage and polyadenylation in vitro. Nucleic Acids Res. 18:5799-5805.
21. Skowronski, J. and M. F. Singer. 1985. Expression of a cytoplasmic LINE-1 transcript is regulated in a human teratocarcinoma cell line. Proc. Natl. Acad. Sci. U.S.A 82:6050-6054.
22. Swergold, G. D. 1990. Identification, characterization, and cell specificity of a human LINE-1 promoter. Mol. Cell Biol. 10:6718-6729.
23. Tabaska, J. E. and M. Q. Zhang. 1999. Detection of polyadenylation signals in human DNA sequences. Gene 231:77-86.
24. Takagaki, Y. and J. L. Manley. 1998. Levels of polyadenylation factor CstF-64 control IgM heavy chain mRNA accumulation and other events associated with B cell differentiation. Mol. Cell 2:761-771.
25. Takagaki, Y., R. L. Seipelt, M. L. Peterson, and J. L. Manley. 1996. The polyadenylation factor CstF-64 regulates alternative processing of IgM heavy chain pre-mRNA during B cell differentiation. Cell 87:941-952.
26. Tchenio, T., J. F. Casella, and T. Heidmann. 2000. Members of the SRY family regulate the human LINE retrotransposons. Nucleic Acids Res. 28:411-415.
27. Thayer, R. E., M. F. Singer, and T. Fanning. Undermethylation of specific LINE-1 sequences in human cells producing a LINE-1-encoded protein. Gene 133, 273-277. 1993.
28. Wallace, A., B. Dass, S. Ravnik, V. Tonk, N. Jenkins, D. Gilbert, N. Copelant, and C. MacDonald. Two distinct forms of the 64,000Mr protein of the Cleavage Stimulation Factor are expressed in mouse male germ cells. 2003.
29. Yue, X., T. Connolly, B. Futcher, and D. Beach. 1991. Human D-Type Cyclin. Cell 65:691-699.
30. Dombroski, B., A. F. Scott, and H. H. Kazazian. 1993. Two additional potential retrotransposons isolated from a human L1 subfamily that contains an active retrotransposable element. Proc. Natl. Acad, Sci. USA. 90:6513-6517.
31. Finnegan, D. J. 1989. The I factor and I-R hybrid dysgenesis in Drosophila melanogaster. In “Mobile DNA (D. H. Berg and M. M. Mowe, eds.); p. 503-517. American Society for Microbiology. Washington, D.C.
32. Schwarz-Sommer, Z., L. Leclercq, E. Gobel, and H. Saedler. 1987. Cin4, an insert altering the structure of the A1 gene in Zea mays, exhibits properties of nonviral retrotransposons. EMBO J. 6:3873-3880.
33. Kimberland, M. L., V. Divoky, J. Prchal, U. Schwahn, W. Berger, and H. Kazazian. 1999. Full-length human L1 insertions retain the capacity for high frequency retrotransposition in cultured cells. Hum. Mol. Genet. 8: 1557-1560
34. Kingsmore, S., B. Giros, D. Suh, M. Beniarz, M. Caron, and M. Seldin. 1994. Glycine receptor beta-subunit gene mutation in spastic mouse associated with LINE-1 element insertion. Nature Genetics 7: 136-141.
35. DeBerardinis, R., and H. Kazazian. 1998. Full-length L1 elements have arisen recently in the same 1-kb region of the gorilla and human genomes. J. Mol. Evol. 47: 292-301.

1. A retrotransposable element wherein one or more polyadenylation sites have been removed from within the coding regions of the genes of said retrotransposable element.

2. A retrotransposable element of claim 1 wherein the element is a non-LTR retrotransposable element.

3. The retrotransposable element of claim 2 wherein the element is a Line-1 element.

4. The retrotransposable element of claim 3 wherein the element is derived from a human, a gorilla, or a mouse.

5. The retrotransposable element of claim 4 wherein the element is Line 1.3.

6. The retrotransposable element of claim 4 wherein the element is Line 1_{RP}.

7. The retrotransposable element of claim 2 having the sequence shown in SEQ ID NO:1.

8. The retrotransposable element of claim 2 having the sequence shown in SEQ ID NO:2.

9. The retrotransposable element of claim 2 having the sequence shown in SEQ ID NO:9.

10. The retrotransposable element of claim 2 wherein the element is an active element.

11. The retrotransposable element of claim 2 wherein the one or more polyadenylation sites are selected from the group consisting of AATAAA, AATACA, AATATA, ACTAAA, AGTAAA, ATTAAA, CATAAA, GATAAA, and TATAAA.

12. The retrotransposable element of claim 2 wherein the strongest predicted polyadenylation site has been removed.

13. The retrotransposable element of claim 2 wherein the strongest predicted polyadenylation site and one or more additional polyadenylation sites have been removed.

14. The retrotransposable element of claim 5 wherein the AATAAA site at position 1788 of SEQ ID NO:3 has been removed.

15. A method for increasing the efficiency of retrotransposition of a retrotransposable element comprising identifying one or more putative polyadenylation sites in the coding region of the element and mutating the one or more putative polyadenylation sites to eliminate their functionality while maintaining the original amino acid coding within the genes of said retrotransposable element.

16. A method for increasing the expression of a gene in a retrotransposable element comprising identifying one or more putative polyadenylation sites in the element and mutagenizing the one or more polyadenylation sites to increase production of mRNA transcripts that comprise sequence located 3′ to the one or more mutagenized polyadenylation sites, while maintaining the original amino acid coding within the genes of the retrotransposable element.
